(19) United States
(12) Patent Application Publication
(10) Pub. No.: US 2005/0119751 A1
(43) Pub. Date: Jun. 2, 2005

(54) Intervertebral bone fusion device

(76) Inventor: Kevin Jon Lawson, Sault Ste. Marie, MI (US)

Correspondence Address: Robert Charles Hill, #821, 235 Montgomery Street, San Francisco, CA, 94104, US

(21) Appl. No.: 10/724,378

(22) Filed: Nov. 28, 2003

(51) Int. Cl. A61B 17/70

(52) U.S. Cl. 623/17.16; 606/61

(57) ABSTRACT

A surgical spine implant comprises a solid wedge shape device body is such that said polygonal shape with decreased height posterior and several surfaces that contours to the anatomy of the spine intervertebral space without complex machining across radiuses. The diminished height posteriorly provides for the angulations necessary to accommodate and ensure maintenance of the normal spinal alignment resulting in posterior extension angulations. The device provides ease of application of bone grafts. Such as for impaction of cancellous bone grafts after placement of the device and narrow posterior aspect easily directs grafts. This minimizes the problem of bone grafts hanging up behind the device either to the right or the left of the medial or lateral, which prevents the displacement of the device or prominence of the bone grafts near the spinal canal. The recess hole in the device accommodates a holding insertion tool and ease of bone graft packing “fill” to ensure a tight fit and immobile the attachment of the disc to the lower vertebrae.

### BACKGROUND OF THE INVENTION

1. Field of the Invention

The present invention relates to surgical methods and devices to treat back and leg pain, and in particular to the surgical insertion of devices that immobilize and promote bone growth within the annulus fibrosis between adjacent vertebrae.

2. Description of Related Art

In the spine, the principal function of the disco-vertebral joint is to transmit compressive loads while allowing flexibility. The adjacent vertebrae are joined with a triple-joint complex. The vertebral bodies form the anterior complex or column. These are shaped like flattened cylinders with disc shaped or oval shaped. The intervertebral discs are sandwiched between each vertebrae. The facet joints in the rear of each vertebrae have a smooth cartilage surface, lubricating joint fluid, and a cover. The facet joints restrict the disc for small degrees of flexion and extension, limit rotation, and protect against translational shear stress. The disc itself comprises two principle parts, the nucleus pulposus at the core, and the annulus fibrosis, which is a multilayer bias-ply wrapping that surrounds the nucleus. The nucleus starts early in life as eighty percent water, and slowly desiccates with age.

A damaged disc can cause nerve dysfunction and debilitating pain in the back, which radiates to legs and arms. Typical treatments that provide relief and allow patients to function again include back braces, medical treatment such as epidural injection or physical therapy. For the severe cases where loss of sensation in arms or legs experienced it requires surgery to remove the disc as the only alternative. Two approaches are used. One replaces the disc and tries to restore joint stability. The other fills the void and promotes bone growth between vertebrae so they will eventually fuse together.

What is needed is a prosthetic bone graft support that can be surgically implanted within the annulus fibrosis to fixate the intervertebral joint and promote bone growth for fusion.

### SUMMARY OF THE INVENTION

An object of the present invention is to provide a bone graft support that corrects spine curvature.

Another object of the present invention is that to provide a prosthetic device that can be inserted with a matching surgical tool.

A further object of the present invention is that to provide a bone graft support that is inexpensive to machine during manufacture.

Briefly, a prosthetic bone graft support embodiment of the present invention comprises a solid device body of biocompatible material intended to be surgically implanted in the spine. It is specially shaped and textured to reduce manufacturing costs, ease surgical insertion, correct spine curvature, and restore stability. The prosthetic nucleus device body has a basic wedge shape for correcting the spine curvature. The top and bottom surfaces have several intersecting planar surfaces that avoid having to machine compound curve radiuses.

An advantage of the present invention is that the device comprises head-tail (cephalad-caudad) with tapering posterior so that the back of the polygon is narrower than the front. This gives greater support anterior to load in preventing subsidence lordosis.

Another advantage of the present invention is that it provides ease of application of bone grafts. Such as for impaction of cancellous bone grafts after placement of the device and narrow posterior aspect easily directs grafts. This minimizes the problem of bone grafts hanging up behind the device either to the right or the left of the medial or lateral, which prevents the displacement of the device or prominence of the bone grafts near the spinal canal.

Further objects, features, and advantages of the present invention will become apparent upon consideration of the following detailed description of specific embodiments thereof, especially when taken in conjunction with the accompanying drawings.

### BRIEF DESCRIPTION OF THE DRAWINGS

FIG. 1 is a diagram representing the spine of a patient with a surgical implant embodiment of the present invention;

FIG. 2 is a perspective view diagram of a pair of surgical implants as they would be positioned within the intervertebral space of the spine of FIG. 1;

FIG. 3 is a perspective view diagram of a surgical implant and a corresponding insertion tool as they would be used in the plain the intervertebral space of the spine of FIG. 1; and

FIG. 4 is a flowchart diagram of a method embodiment of the present invention.

### DETAILED DESCRIPTION OF THE INVENTION

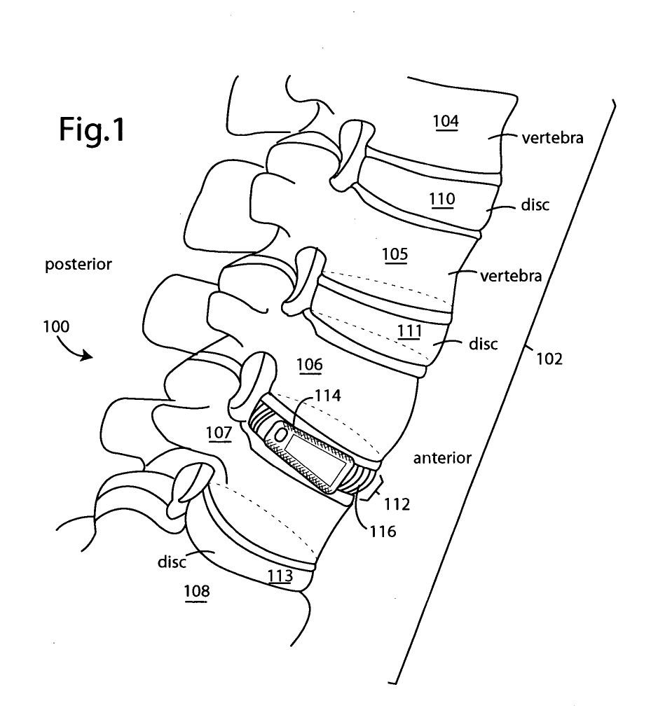

FIG. 1 illustrates the placement of a surgical implant embodiment of the present invention, referred to herein by the reference numeral 100. A human spine 102 commonly comprises a series of vertebrae 104-108 interdigitated with a corresponding series of discs 110-113. Each natural disc comprises a nucleus pulposus surrounded and contained by a corresponding annulus fibrosis. Natural nucleus pulposus have jelly-like structures that can absorb and dampen compressive shock loads. Natural annulus fibrosis structures comprise multiple layers of bias-ply filaments set at forty-degree angles that resemble the construction of an automobile bias-ply tire carcass.

Disc 112, between vertebra 106 and 107, is assumed in FIG. 1 to be degenerated. A surgical implant embodiment of the present invention, referred to herein by reference numeral 114, is surgically embedded in the inter-vertebral space between vertebra 106 and 107, and inside an annulus fibrosis 116.

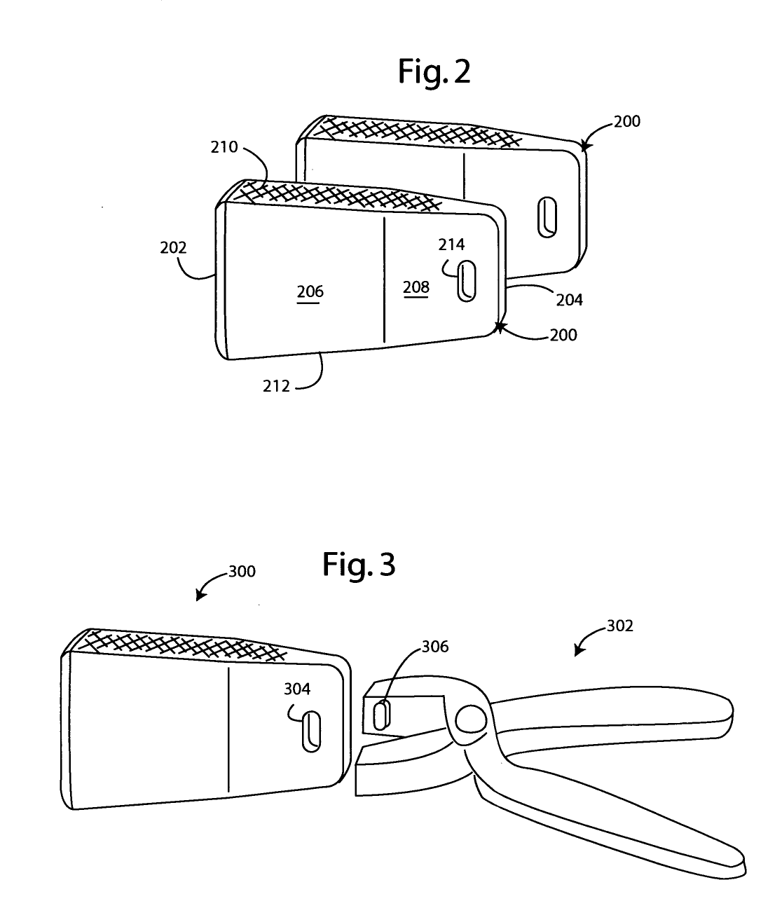

FIG. 2 represents a pair of surgical implants 200 like that of FIG. 1. These are used in pairs between adjacent vertebrae to stabilize and immobilize the affected joint. Biocompatible materials are used, e.g., high molecular weight plastics. The purpose of surgical implants 200 is to promote the growth of bone between the vertebrae that will ultimately fuse them together.

Each surgical implant 200 has a nose or head-end 202 and a tail-end 204. Two parallel opposing left and right middle sides are represented by a side 206. Two opposing tapering tail sides are represented by tail side 208. These help accommodate an insertion tool, and ease bone graft packing fill. The tapering posterior makes it easier to pack-in cancellous bone grafts on both sides medial and lateral to each of the surgical implants 200 after initial placement.

Opposing top and bottom surfaces 210 and 212 act as intervertebral spacers and are textured to reduce slippage at the contact with the vertebral faces. Such can be diamond or checkered texturing. The top and bottoms 210 and 212 should have a tapering cross section toward the tail-end 204 to provide for correction of the spinal tilt. Such lordotic segment provides for the angulation required for normal spine alignment by giving greater anterior support. A gripping-tool hole 214 allows a surgical insertion tool to firmly lock on and hold the surgical implants 200. The taper in the device body allows such tool to be used for an incision only large enough to accommodate the largest cross section of the device body.

The enveloping sides comprise planar surfaces that have been eased at their intersections to remove sharp edges. Such planar surfaces are easier and less expensive to manufacture that are compound radiused surfaces.

FIG. 3 represents a surgical implant 300 like that of FIGS. 1 and 2. A surgical insertion tool 302 is provided for locking onto a gripping hole 304 with a matching tooth 306. The surgical insertion tool 302 resembles a pair of ordinary pliers. A typical surgical implant 300 will be 6-9 millimeters in width, 10-16 millimeters in height, and about 22 millimeters long.

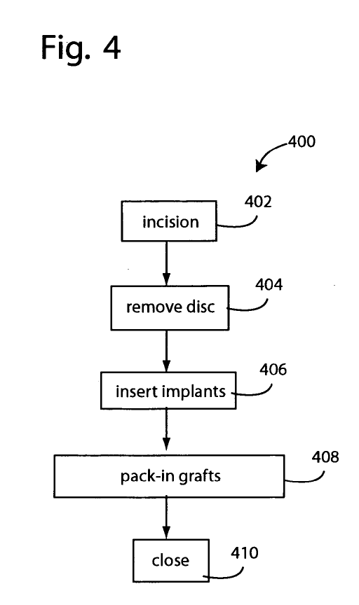

FIG. 4 represents a method embodiment of the present invention, and is referred to herein by general reference numeral 400. The method 400 provides for surgically implanting a prosthetic in a human spine to promote bone fusion of two adjacent vertebrae. For example, the surgical implants illustrated in FIGS. 1-3. A step 402 makes a flap technique incision of an annulus fibrosis corresponding to an affected area of a spine. A step 404 removes the diseased or deteriorated disc. A step 406 inserts two surgical implants through the incisions in the annulus fibrosis. A step 408 packs bone grafts and between and lateral to the surgical implants. A step 410 closes the incision in the annulus fibrosis when feasible. A permanent bone growth and fusion between the inferior and superior vertebrae then occur naturally after surgery.

Although particular embodiments of the present invention have been described and illustrated, such is not intended to limit the invention. Modifications and changes will no doubt become apparent to those skilled in the art, and it is intended that the invention only be limited by the scope of the appended claims.

1. A surgical implant, comprising:
a device body having a head-end and a tail-end, and overall with a relatively narrow width, and a relatively taller height for insertion between adjacent upper and lower vertebrae;
an indent in said tail end providing for a secure gripping of the device body with a tool during surgical implantation;
a set of intersecting and symmetrical planar surfaces enveloping the device body and providing for simplified machining compared to compound radius surfacing;
a taper between said head and tail ends comprising two opposing ones of said set of intersecting and symmetrical planar surfaces and providing for a relative front-back tilt between said adjacent upper and lower vertebrae; and
a textured surface disposed on said two opposing ones of said set of intersecting and symmetrical planar surfaces.

2. The implant of claim 1, wherein:
the device body is about 6-9 millimeters in width, 10-16 millimeters in height, and about 22 millimeters long.

3. The implant of claim 1, wherein:
the indent is a hole that fits and matches a corresponding tooth in said tool.

4. The implant of claim 1, wherein:
the set of intersecting and symmetrical planar surfaces are configured to minimize manufacturing costs.

5. The implant of claim 1, wherein:
the taper is oriented posteriorly in a patient and provides for easier packing of cancellous bone grafts and around and between a pair of implants.

6. The implant of claim 1, wherein:
the taper allows said tool to be used for an incision only large enough to accommodate the largest cross section of the device body.

7. A method for surgically implanting a prosthetic in a human spine to promote bone fusion of two adjacent vertebrae, comprising:
a flap technique incision of an annulus fibrosis corresponding to an affected area of a spine;
removing a diseased or deteriorated disc;
inserting two surgical implants through the incisions in the annulus fibrosis; and
packing bone grafts and between and lateral to said surgical implants;
wherein, permanent bone growth and fusion between inferior and superior vertebrae then occur naturally after surgery.

8. The method of claim 7, further comprising:
closing said incision in the annulus fibrosis after the step of packing bone grafts.
